(19) United States
(12) Patent Application Publication
(10) Pub. No.: US 2018/0235837 A1
(43) Pub. Date: Aug. 23, 2018

(54) FILLING DEVICE FOR FILLING A MEDICAL BAG, METHOD FOR PRODUCING SUCH A DEVICE AND PLANT FOR PRODUCING FLUID-FILLED MEDICAL BAGS

(71) Applicant: KIEFEL GMBH, Freilassing (DE)

(72) Inventor: Martin Klein, Hallein (AT)

(21) Appl. No.: 15/750,232

(22) PCT Filed: Jul. 7, 2016
(86) PCT No.: PCT/DE2016/000265

§ 371 Date: Feb. 5, 2018

(30) Foreign Application Priority Data
Oct. 15, 2015 (DE) 10 2015 013 259.8
May 10, 2016 (DE) 10 2016 005 596.0

(51) Int. Cl. A61J 1/10; A61M 39/10; B65D 83/42

(52) U.S. Cl. CPC A61J 1/10; A61M 39/105; B65B 3/003; A61M 2207/00; B65D 83/425

(57) ABSTRACT

To further develop filling devices for filling a medical bag, in particular in terms of a massive basic body or massive filling blocks, proposes the invention a filling device for filling a medical bag with fluids comprising a basic body with fluid channels and valve seats, on which valve means can be arranged to adjust fluid volume flows passing through fluid channels, wherein the basic body is produced at least partially by means of an additive producing process.

The invention relates to a filling device for filling a medical bag with fluids comprising a basic body with fluid channels and valve seats on which valve means can be arranged to adjust flowing fluid volume flows passing through the fluid channels.

The invention relates to a method for producing a filling device for filling a medical bag in which a basic body of the filling device is equipped with fluid channels for conducting fluids which are to handle for the medical bag and with valve seats for adjusting fluid volume flows.

The invention also relates to a plant for producing medical bags filled with fluids with a device for producing the medical bags, with a device for filling the produced medical bags and with a device for closing the filled medical bags.

Filling devices of the related art in particular are known from the prior art. These filling devices of the related art include usually a basic body which can carry one or more filling needles, that can be inserted in an access port of the medical bag. Furthermore, the basic body is penetrated with fluid channels, with which in particular fluids are guided into the medical bag from a thereto running supply pipe to the correspondent filling needle. A release or closure of said fluid channels is arranged usually via diaphragm valves, which are attached on the basic body of the filling device. In this respect, the basic body has one or more valve seats, on which the diaphragm valves are arranged in such a way that with them the corresponding fluid channel can be closed or can be released or opened.

The valve seat or the diaphragm valve sits here either somewhere on the track of the respective fluid channel, or in the immediate vicinity of the filling place for the medical bag or in the immediate vicinity—seen in the flow direction—before the filling needle. Normally the latter variant is preferable because considerably less dead space between the diaphragm valve or valve seat and the filling place or the filling needle is available, so that the filling of the medical bag can be controlled better. In this respect, on the basic body both the valve seats and the filling needle required fluid media connections for connecting the thereto running supply pipe and just the corresponding fluid channels between with these components are integrated.

A filling device constructed in this way, in which a basic body equipped in this way is preferably cyclically supplied to a port of a medical bag to be filled or removed from this, has proven well in practice.

However, in the context of optimized processes or plants for manufacturing, filling and deploying filled medical bags are expected higher and higher clock rates, so that the clock times of the filling device must be improved or optimized accordingly.

In addition, increasing demands on the filled bag also require higher requirements to the filling device or in particular to the basic body, since the medical bags must often be additionally evacuated in a different sequence and/or must be filled with gas.

This means in total that for such a gas filling or evacuation additional fluid pipes and diaphragm valves or valve seats have to be integrated in the basic body, whereby the basic body gets always larger and thus must be build heavier.

Larger dimensions and especially higher weights have a negative effect on the achievable clock rates or cycle times.

The invention is therefore based on the object, to improve filling devices of the related art in such a way that the clock rates with regard to the filling device are optimized so that at least the filling device is as small as possible and is no decisive limiting factor in a plant for the production of medical bags, even if the basic body is equipped with additional fluid channels and valve seats or the like.

The object of the invention is solved according to a first aspect of a filling device for filling a medical bag with fluids comprising a basic body with fluid channels and valve seats, on which valve means can be arranged to adjust fluid volume flows passing through the fluid channels, wherein the filling device is characterized in that the basic body is at least partially produced by means of an additive producing process.

At present, such basic bodies are produced with conventional subtractive producing processes, such as milling, turning or eroding. With increasing complexity of the basic body it becomes more difficult to produce a related art basic body economically. In addition, it is particularly difficult, to bring several fluid channels in a close type space in the basic body, with the result that such basic bodies are build getting larger. As a result, however, more masses must be accelerated, which in turn is negatively on achievable cycle times with regard to the filling device for filling medical bags.

In contrast to the conventional subtractive producing processes, it succeeds by means of additive producing process to provide the basic body particularly compact even if on the basic body a plurality of additional fluid channels for example for gas filling or evacuation of the medical bag are present.

Such additive manufacturing methods are characterized in contrast to classical subtractive producing processes, such as milling, drilling, turning, eroding or the like, in particular in that materials for the production of a component can be added for example essentially layer by layer. By such additive producing processes available producing processes may also be preferably completely eliminated, namely those which are based on joining methods such as welding, soldering or the like, for example, to attach a connection flange for fluid supply pipes or the like to the basic body.

In the present case, almost any additive producing process can be used, as later described as an example later.

In any case, the basic body of the filling device for filling a medical bag can, by means of such an additive producing process, be built substantially more compact and thus lighter weight, making this body also significantly better to accelerate. This in turn can provide with the present filling device also higher cycle times at a corresponding equipped plant for producing and filling medical bags of the related art.

The term “basic body” describes in this case a versus a medical bag movable, solid build filling block element which represents, at least partially, the fluid channels and the valve seats. In the sense of the present invention, the terms basic body and filling block element can be used synonymously.

The term “cycle times” describes in the context of the invention processes in which the basic body of the filling device for the purpose of filling or evacuating a medical bag is brought close to this medical bag, then on the medical bag filling and/or evacuation operations, and after completion of such filling or evacuation operations of the basic body is removed from the thus treated medical bag, so that a further medical bag can be supplied in particular to the filling block on the filling device.

The object of the invention is furthermore also solved according to a further aspect by a filling device for filling a medical bag with fluids comprising a basic body with fluid channels and valve seats, to which valve means can be arranged to adjust fluid volume flowing through the fluid channels there through, wherein the filling device is characterized in that within the basic body running fluid channels are at least partially bow-shaped designed.

By the fact that the relevant fluid channels are designed bow-shaped, they can passed running each other within the basic body significantly narrower, so that for this reason the basic body can be made more compact, which in turn advantageously enables a weight reduction, so that with regard to the filling device fewer masses have to be accelerated. As a result, with respect to the filling device also shorter cycle times are achieved.

For example, parallel or skewed to each other or intersecting fluid channels less than 10 mm, preferably less than 5 mm from each other are spaced arranged on the basic body.

It is particularly advantageous that the corresponding bow-shaped fluid channels can be formed edgeless in contrast to the prior art, so that in such a way curved or bended fluid channels with such curves can be significantly flowed through with less turbulence, resulting in shorter filling times. This also succeeds an optimization of the cycle times in the context of the invention.

So far, such fluid channels have been introduced through holes in the basic body, whereby two holes must penetrate to can form a corresponding curve on a fluid channel.

However, if two drill holes or holes run into each other on the basic body in such a way that the fluid channel is not just running straight, it always comes to an edge formation, at which turbulences result in a fluid volume flow which flows through the fluid channel, whereby the filling times worsen. But this is excluded in the present case.

In general, with the proposed solutions regarding the fluid volume flows unfavorable tear-off edges can be avoided within the fluid channels.

In addition, both solutions allow accommodating additional fluid channels in the basic body in a compact manner, thereby using the present produced basic body further functions of the filling device, such as a bag control etc. can be structurally enabled very easy.

With this body all the essential functions with respect to a filling device can be realized in a filling block element, such as filling, blowing to create a headspace, vacuum drawing or the like concerning the medical bag.

In this respect, a preferred embodiment provides that at least one of the fluid channels is configured continuously curved. Through a continuously curved fluid channel an edge formation within the fluid channel can be prevented.

In this respect, the fluid channels provided on the basic body are in the present case preferred also entirely free of edges or free of stall.

In general, this also allows a reliable complete emptying of the fluid channels, for example, after cleaning, so the risk of contamination by dirt particles or the like of fluids to be brought in particular in the medical bag can be further reduced significantly.

Furthermore, it is advantageous if at least one of the fluid channels with respect to its total length is designed curved more than 50%, preferably more than 80%.

While with conventional subtractive manufacturing techniques only angular fluid channels can be made, it is advantageous in the present case to design a curve on the fluid channel, if the curve is designed with a larger radius.

Thus, it is possible that the fluid channels are designed significantly streamlined, whereby the advantages already mentioned above can be achieved.

A fluid channel provided on the basic body can be designed more streamlined when the length of a curved portion of one of the fluid channels is more than 50%, preferably more than 100%, of the length of a straight section of this fluid channel.

If at least one of the fluid channels along its longitudinal extension comprises a variable cross-section, further advantageous flow effects can be achieved with respect to the fluids at the filling device.

By means of such a variable inner diameter, for example different flow rates within one fluid channel provided on the basic body can be achieved.

Preferably, the variable cross section widens continuously or constant.

For example, the variable cross section relates to the total length of the respective fluid channel.

Or the variable cross section refers only to a section of the fluid channel, wherein the subsection, for example, is more than 30% or more than 50%, preferably more than 80%, of the total length of the affected fluid channel.

For example, at least one of the fluid channels is designed in sections conically, whereby within a fluid channel approximately different pressures or flow rates can be achieved without operating a valve means associated with this fluid channel.

In addition, a weight reduction on the basic body can also be achieved when at least one of the fluid channels is protruding from a side wall of the basic body.

Here, a fluid channel section protruding over the side wall of the basic body is then no longer surrounded by the entire basic body, so that only with this a significant weight reduction can be already achieved.

In addition, at the filling device, in particular with regard to the basic body further weight reduction can be achieved if between fluid channels existing gaps are at least partially free of material or are component-free.

Such material-free or component-free gaps between individual fluid channels can be achieved in many different ways. However, it is especially easy to produce such gaps in an additive producing process.

Anyway, with regard to the basic body almost any pipe guidance of the fluid channels can be achieved when the fluid channels are produced at least partially by means of an additive producing process.

Also valve seats for corresponding valve means can be produced on the basic body particularly simple and therefore cost-efficient, if the valve seats are produced at least partially by means of an additive producing process.

Are in addition fastening means for attaching valve means on the basic body at least partially produced by means of an additive producing process, the production of the basic body or the present filling device can be further simplified.

It is also expedient if fluid inlets and/or fluid outlets are at least partially produced by means of an additive producing process.

The term “fluid inlets” describes in the context of the invention, for example, a connection to the basic body, on which by means of fluid supply pipes or the like the filling device can be connected to the basic body.

The term “fluid outlets” describes in the context of the invention, in contrast, connection options, on which, for example, filling needles or like of the filling device can be attached to the basic body.

Better flow conditions can be created on the basic body, when the fluid facing surfaces of the fluid channels, of the valve seats, of the fluid inlets and/or fluid outlets are produced at least partially by means of an additive producing process.

Especially, to easy manufacture structurally over the basic body protruding fluid channels around over the side wall, it is advantageous if the fluid channels are partially configured through pipe segments, which protrudes over side walls of the basic body, whereby said pipe segments and the basic body are produced together at least partially by means of an additive producing process.

The basic body of the present filling device can be made even more compact when the basic body comprises connection means for connecting fluid inlets or filling elements to change interact with the medical bag, whereby said connection means and the basic body are at least partially produced together by means of an additive producing process.

The basic body of the present filling device can be built even more compact when multiple connection means are arranged to a common support bar element side by side, whereby this common support bar element and the basic body are at least partially produced together by means of an additive producing process. Are several connection means arranged on the common support bar element, can these connection means be also very stable and be provided summarized on the body.

The common support bar element can in this case be alone constructively connected on the basic body by protruding pipe segments from side walls of the basic body.

Conveniently, the common support bar element is arranged on the basic body by means of an additional holder part, whereby said additional holder part, said support bar element and said basic body are together at least partially produced by means of an additive producing process.

Due to the additional holder part, the support bar element can also be independent of the strength of the pipe segments reliably arranged and be held on the basic body.

Here are also other combinations of generatively produced areas of the basis body possible.

As already mentioned, it is understood that for the purposes of the invention different additive producing processes can be used.

In the present case, it has been found that the basic body can be produced particularly advantageous if the additive producing process is a 3D printing process, a 3D laser sintering method or the like.

In particular, laser melting methods (SLM methods) are also suitable also to be used in the context of the invention.

A preferred embodiment therefore also provides that the basic body is made of formless or shape-neutral materials.

In the present case, the term “basic body” refers in particular to fluid channels, valve seats, fluid inlets, fluid outlets, fastening means for attachment of valve means, over side walls projecting pipe segments and/or previously mentioned connection means or the like.

Formless materials are for the purposes of the invention, for example, liquids, powders or the like, wherein form neutral materials are executed band, wire or the like.

With regard to the above-mentioned 3D printing method can procedural be differentiated between powder bed processes, free space processes and liquid material processes.

Powder bed processes include, for example, selective laser melting (SLM), selective Laser sintering (SLS), selective heat sintering (SHS), binder jetting (solidification of powder material by means of binder) as well as electron beam melting (electron beam melting=EBM).

The free space processes include fused depositing modeling (FDM) or else fused filament fabrication (FFF), laminated object modeling (LOM), welding by order or cladding, wax deposition modeling (WDM), contour crafting, cold injection molding and electron beam welding (electron beam welding=EBW).

Liquid material processes include, for example, stereolithography (SLA) and Micro-SLA, digital light processing (DLP) and liquid composite molding (LCM).

In this respect, the object of the invention is also solved by a method for producing a filling device for filling a medical bag, in which a basic body of the filling device is provided with fluid channels for conducting fluids to handle with respect to the medical bag and with valve seats for adjusting fluid flow rates, wherein the method is characterized in that the basic body is produced at least partially generative.

The basic body of the present filling device can be made particularly simple, i.e. both structurally and procedurally simple, with qualitatively very high class fluid channels when the basic body is generated successively at least around the fluid channels.

A further variant of the method provides that the basic body, the fluid channels, the valve seats and/or fluid inlets and fluid outlets disposed on the basic body are produced by a single producing process, so that the production in particular of the basic body of the present filling device is significantly simplified with a single production method.

If the basic body is produced at least partially from a data record for operating a single manufacturing plant, the preparation of the filling device can be further simplified considerably.

Preferably, only a single data record is used to produce the basic body in which any information for producing the fluid channels, the valve seats, etc. in the basic body are included.

In addition, the object of the invention is solved with a plant for producing of fluid filled medical bags with a device for producing the medical bag, with a device for filling the produced medical bags and with a device for closing the filled medical bags, whereby the plant is in particular characterized by a filling device according to one of the features described here.

By means of the present invention can almost be made possible any design and wiring with respect to the fluid channels within or on the body. In this respect, there are significantly fewer restrictions of complexity, which still exists in the previously used subtractive producing processes.

In addition, it is advantageous that all the necessary valve means can be arranged in smallest space directly on the body.

Furthermore, most streamlined design options of the present fluid channels or the like are easily possible.

It is particularly advantageous that the fluid channels are formed as short as possible in particular by their curved or bow-shaped configuration.

Additive producing processes already require very high surface finishes, which is very beneficial especially on fluids interacting surfaces.

With corresponding reworking even higher surface finishes can be achieved.

The intermittent docking or undocking of filling elements, like in particular filling needles, can be done quickly at the plant by means of the presently configured filling device in particular with regard to the advantageously designed basic body, because much smaller masses must be moved with respect of the present basic body, whereby related cycle times can be reduced significantly.

Other features, effects, and advantages of the present invention will become apparent with attached drawing and subsequent description, in which by way of example a filling device, in particular with regard to its by an additive producing process produced or generated basic body is shown and described.

In the drawing show:

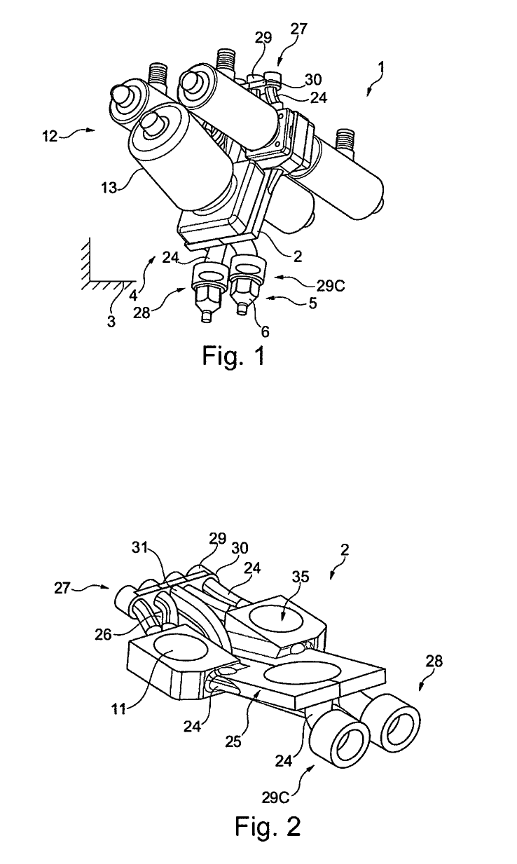

FIG. 1 a schematic perspective view of the filling device;

FIG. 2 schematically a perspective view of the basic body;

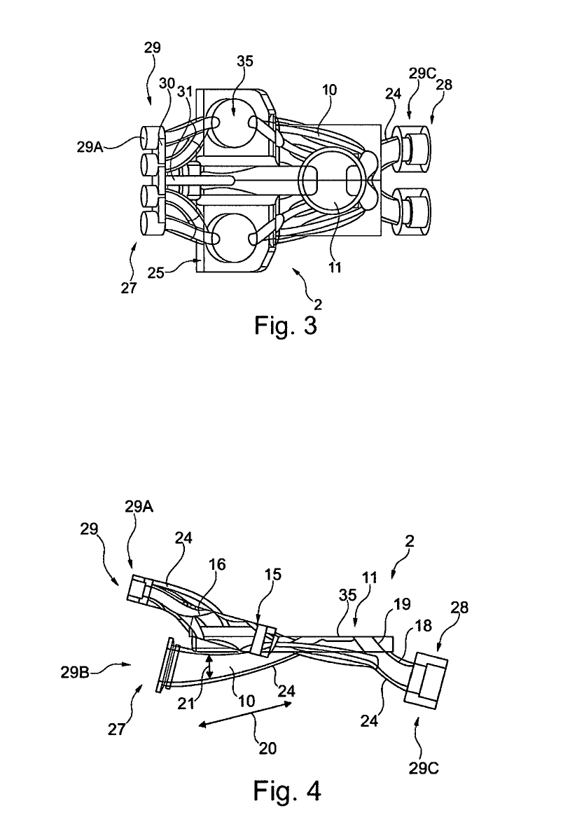

FIG. 3 schematically a top view of the basic body that is shown in FIGS. 1 and 2; and

FIG. 4 schematically a partially sectioned side view of the basic body shown in FIGS. 1 to 3.

According to the representation according to FIG. 1, a filling device 1 for filling at least partially a medical bag (not shown here) is shown in particular in terms of their with respect to this medical bag intermittently displaceable basic body 2 at a plant 3 for producing medical bags filled with fluids.

The basic body 2 of the filling device 1 provides in the context of the invention massive filling block 4, which is linearly displaceable for docking of filling elements 5 to the medical bag to be filled with respect to this medical bag or a corresponding bag holding device (not shown).

The filling elements 5 are in this embodiment as filling needles elements 6 performed.

The basic body 2 or the massive filling block 4 has a plurality of fluid channels 10 (only exemplified numbered, see in particular FIGS. 3 and 4) and valve seats 11 (only exemplified numbered) on which valve means 12 (only numbered as an example) are arranged.

These valve means 12 are performed in this embodiment as diaphragm valves 13 (numbered only as an example), wherein at the present basic body 2 a total of five such valve means 12 and diaphragm valves 13 respectively are arranged as well visible according to the illustration of FIG. 1.

In this respect, on the basic body 2 fastening means are available (not explicit here shown) for securing the valve means 12, whereby such fastening means can be of different nature.

For example, such fastening means include threaded holes, in which screws of the valve means 12 can be screwed to attaches the valve means 12 to the basic body 2.

Furthermore, such fastening means may have centering means, by means of which defines the valve means 12, for example secured against rotation, on that the basic body 2 can be arranged.

In any case, the basic body 2 is produced by means of an additive producing process, wherein this basic body 2 is produced by means of a 3D laser sintering method, whereby in particular the fluid channels 10 are arranged very compact side by side within the basic body 2 and generally on the basic body 2.

As a result of this dense side by side position of the fluid channels 10, the basic body 2 is build particularly compact, so lower masses in terms of docking and undocking the medical bag must be moved, which in turn allows to shorten decisively cycle times.

This makes it possible to produce medical bags to be filled with fluids on the plant 3 much more effective, since by the filling device 1 the operating clocking timing between the device for producing the medical bags and the device for closing the filled medical bags, etc. is ideally not limited.

In addition, the fluid channels 10 can be much easier introduced in the basic body 2, since an otherwise required subtractive producing process such as for example, drilling, can be completely eliminated.

As a result, the fluid channels 10 can also be optimized for flow, since they can be produced edge-free by means of the additive producing process in the area of curves 15 (only explicitly numbered), as clearly recognizable for example in accordance with the illustration of FIG. 4.

It is particularly advantageous, that in particular within the basic body 2 extending fluid channels 10 are configured bow-shaped, that is curved, whereby fluids to be passed through the fluid channels 10 are again more optimized to pass through the fluid channels 10.

Only, for example, this is well illustrated with respect to the fluid channel curve section 16, since the fluid channel 10 is configured continuously curved in this fluid channel curve section 16.

This is all the more true if at least one of the fluid channels 10 is configured with respect to its total length (not explicitly drawn here) more than 50% or better even more then 80% curved, in particular continuously curved, so that as a result fluid channels 10 can be realized bent with large radii, causing that the fluids passing thought the fluid channels configured in this way are guided particularly turbulence-free up to the filling elements 5.

Preferably, the fluid channels 10 are bow-shaped over their entire length, that is curved, designed, whereby the individual fluid channels can be realized even with more complicated geometries dense or very close to each other on the basic body 2, whereby it can be provided naturally very compact and therefore with a lesser mass.

For example, the length of a curved section 18 is more than 100% of the length of a straight portion 19 of this fluid channel 10, as is only numbered shown in terms of FIG. 4 by way of example.

In addition, the basic body 2 comprises at least one fluid channel 10, which has along its longitudinal extension 20 (see for example FIG. 4) a variable cross section 21.

In other words, this means that fluid channels 10 on the basic body 2 have a variable inner diameter.

For example, this is a constantly changing cross section 21, wherein the relevant fluid channel is extended continuously funnel-shaped—approximately in basic flow direction of the respective fluid.

In the present case, it does not matter if the fluid channels 10 are arranged entirely within the basic body 2 or alternatively at least partially outside of the basic body 2. Depending on the variant, it is also possible that at least one of the fluid channels 10 is arranged extending completely outside the basic body 2.

Preferably, however, the fluid channels 10 are both partially within the body 2 disposed as well as partially disposed outside of the basic body 2, whereby in particular here the respective fluid channel 10 protrudes from one of the side walls 25 (numbered only as an example) of the basic body 2.

For completeness, it should be explained here that the term “side walls” comprises in general any outer boundary (not separately numbered here) of the basic body 2, so it describes front, back, upper and lower sides and other sides of the basic body 2.

Furthermore, it is with respect to a weight reduction of the basic body 2 so that between individual fluid channels 10 existing gaps 26 (only explicitly numbered) are configured material-free, so that with respect to the basic body 2 to accelerating masses are again significantly reduced.

The basic body 2 shown in FIGS. 1 to 4 is with regard to its fluid channels 10 produced entirely by means of an additive producing process.

Also, all the valve seats 11 are on the basic body 2 produced by means of the additive producing process.

Even the not shown fastening means of the valve means 12 are produced by the additive producing process.

In addition, the base body 2 comprises at least in this embodiment a total of five fluid inlets 27 (only explicitly numbered) and a total of two fluid outlets 28, which are produced also all by means of the additive production process.

The fluid inlets 27 and the fluid outlets 28 embody here in general connection means 29 of the basic body 2

Of these, at least four of these fluid inlets 27 are performed as upper connection means 29A, which are arranged on a common support bar element 30 side by side.

The support bar element 30 is for stability reasons attached by means of an additional holder part 31 additionally to the basis body 2. In this respect, this results in a very solid and compact connection unit.

The connection means 29 serve in this context to connect fluid supply pipes, whereby the connection means 29A or fluid inlets 27 are arranged on the common support bar element 30 and provided for gaseous fluids, and connection means 29B arranged below the support bar element 30 are provided for a liquid fluid.

Also, the fluid inlets 27 and the fluid outlets 28 and all connection means 29 related thereto are produced by means of the additive producing process.

Thus, preferably, all the surfaces 35 facing the fluids (here only exemplified in terms of the valve seats 11) are produced also by means of the additive producing process.

At least in this embodiment, the basic body 2 is completely produced via the additive producing processes, making it both constructive and procedurally particularly effective and therefore also produced cost-efficiently.

It should be explicitly stated at this point that the features of the present or in the claims and/or in the figures described solutions can be combined to achieve correspondingly cumulative the explained features, effects and benefits.

It is understood that the example described above is only to a first embodiment of the filling device 1 according to the invention and in particular of their basic body 2 respectively. In this respect, the execution of invention is not limited by the design of this embodiment.

All features disclosed in the application documents are claimed as essential to the invention, if they are individually or in combination new compared with the prior art.

### LIST OF REFERENCE SIGNS USED

1 filling device

2 basic body

3 plant

4 massive filling block or filling block element

5 filling elements

6 filling needle elements

10 fluid channels

11 valve seats

12 valve means

13 diaphragm valves

14 continuously curved bow

15 curve

16 fluid channel curve section

18 curved section

19 straight section

20 longitudinal extension

21 cross section

24 pipe segments

25 side walls

26 gaps

27 fluid inlets

28 fluid outlets

29 connection means

29A upper connection means

29B lower connection means

29C additional connection means

30 common support bar element

31 additional holder part

35 surfaces facing the fluids

We claim:

1. Filling device (1) for filling a medical bag with fluids comprising a basic body (2) with fluid channels (10) and valve seats (11), on which valve means (12) can be arranged to adjust fluid volume flows passing through the fluid channels (10), characterized in that the basic body (2) is at least partially produced by means of an additive producing process.

2. Filling device (1) for filling a medical bag with fluids comprising a basic body (2) with fluid channels (10) and valve seats (11), on which valve means (12) can be arranged to adjust fluid volume flows passing through the fluid channels (10), characterized in that within the basic body (2) running fluid channels (10) are at least partially bow-shaped (14) designed.

3. Filling device (1) according to claim 2, characterized in that at least one of the fluid channels (10) is designed steady curved.

4. Filling device (1) according to claim 2, characterized in that at least one of the fluid channels (10) is with respect to his total length designed more then 50% curved, especially more that 80%.

5. Filling device (1) according to claim 2, characterized in that the length of a curved portion (18) of one of the fluid channels is more than 50%, preferably more than 100%, of the length of a straight section (19) of this fluid channel (10).

6. Filling device (1) according to claim 2, characterized in that at least one of the fluid channels (10) along its longitudinal extension (20) comprises a variable cross-section (21).

7. Filling device (1) according to claim 2, characterized in that at least one of the fluid channels (10) is protruding from a side wall (25) of the basic body (2).

8. Filling device (1) according to claim 2, characterized in that between fluid channels (10) existing gaps (26) are at least partially free of material or are component-free.

9. Filling device (1) according to claim 2, characterized in that the fluid channels (10) are produced at least partially by means of an additive producing process.

10. Filling device (1) according to claim 2, characterized in that the valve seats (11) are produced at least partially by means of an additive producing process.

11. Filling device (1) according to claim 2, characterized in that the fastening means for attaching valve means (12) on the basic body (2) are at least partially produced by means of an additive producing process

12. Filling device (1) according to claim 2, characterized in that fluid inlets (27) and/or fluid outlets (28) are at least partially produced by means of an additive producing process.

13. Filling device (1) according to claim 2, characterized in that the fluid facing surfaces (35) of the fluid channels (10), of the valve seats, of the fluid inlets (27) and/or fluid outlets (28) are produced at least partially by means of an additive producing process.

14. Filling device (1) according to claim 2, characterized in that the fluid channels (10) are partially configured through pipe segments (24), which protrudes over side walls (25) of the basic body (2), whereby said pipe segments (24) and the basic body (2) are produced together at least partially by means of an additive producing process.

15. Filling device (1) according to claim 2, characterized in that the basic body (2) comprises connection means (29, 29A, 29B, 29C) for connecting fluid inlets or filling elements (5) to change interact with the medical bag, whereby said connection means (29, 29A, 29B, 29C) and the basic body (2) are at least partially produced together by means of an additive producing process.

16. Filling device (1) according to claim 2, characterized in that multiple connection means (29, 29A) are arranged to a common support bar element (30) side by side, whereby this common support bar element (30) and the basic body (2) are at least partially produced together by means of an additive producing process.

17. Filling device (1) according to claim 2, characterized in that the common support bar element (30) is arranged on the basic body (2) by means of an additional holder part (31), whereby said additional holder part (31), said support bar element (30) and said basic body (2) are together at least partially produced by means of an additive producing process.

18. Filling device (1) according to claim 2, characterized in that the additive producing process is a 3D printing process, a 3D laser sintering method or the like.

19. Filling device (1) according to claim 2, characterized in that the basic body is made of formless or shape-neutral materials.

20. Method for producing a filling device (1) for filling a medical bag, in which a basic body (2) of the filling device (1) is provided with fluid channels for conducting fluids to handle with respect to the medical bag and with valve seats (11) or adjusting fluid flow rates, characterized in that the basic body (2) is produced at least partially generative.

21. Method according to claim 20, characterized in that the basic body (2) is generated successively at least around the fluid channels (10).

22. Method according to claim 20, characterized in that the basic body (2), the fluid channels (10), the valve seats (11) and/or fluid inlets (27) and fluid outlets (28) disposed on the basic body (2) are produced by a single producing process (producing type).

23. Method according to claim 20, characterized in that the basic body (2) is produced at least partially from a data record for operating a single manufacturing plant.

24. Plant for producing of fluid filled medical bags with a filling device (1) or producing the medical bags, with a device for filling the produced medical bags and with a device for closing the filled medical bags characterized by a filling device (1) according to claim 1.
